(19) United States
(12) Patent Application Publication
(10) Pub. No.: US 2025/0302527 A1
(43) Pub. Date: Oct. 2, 2025

(54) DEVICE AND METHOD FOR THE IRREVERSIBLE ELECTROPORATION OF TISSUE

(71) Applicant: Stockert GmbH, Freiburg im Breisgau (DE)

(72) Inventors: Fabian Kees, Freiburg im Breisgau (DE); David Stork, Freiburg (DE)

(21) Appl. No.: 19/094,028

(22) Filed: Mar. 28, 2025

(30) Foreign Application Priority Data
Mar. 28, 2024 (DE) 102024108889.3

(51) Int. Cl. A61B 18/14; A61B 18/00

(52) U.S. Cl. CPC A61B 18/1492; A61B 2018/0022; A61B 2018/00613; A61B 2018/00642; A61B 2018/00875; A61B 2018/00904; A61B 2018/1467

(57) ABSTRACT

A device and method for the irreversible electroporation of tissue. An exemplary embodiment of the device has a catheter and a signal generator-evaluation unit connected to the proximal end of the catheter.

### CROSS-REFERENCE TO RELATED APPLICATIONS

This application claims priority to German Patent Application Serial No. DE 10 2024 108 889.3 filed Mar. 28, 2024.

### BACKGROUND

### Field

A device and a method for the irreversible electroporation of tissue are presented here.

### Discussion of the Related Art

In recent years, the treatment of tissue by means of pulsed electrical fields has become established as an increasingly relevant clinical technique. The use of short high-voltage pulses and the high electrical field strengths associated therewith, which act on the tissue, have, however, already been the subject of intensive research for more than four decades. This application method is categorised as a non-thermal procedure, since it is based on the delivery of short pulses with a high voltage amplitude, which generate between active electrode pairs a locally strong electrical field in the region of up to several hundred volts per centimetre. This field strength leads to the formation of pores in the cell membranes. If the electrical field exceeds a specific threshold value, which is required for the formation of pores in the lipid bilayers of the cell membranes, and if the tissue is exposed to that field over a critical period of time, the electroporation becomes irreversible. The pores remain permanently open, which ultimately leads to programmed cell death (apoptosis) of the cell in question.

Irreversible electroporation (IRE) is primarily a non-thermal procedure, which effects only a slight increase in the tissue temperature by several degrees for a few milliseconds. This distinguishes it significantly from conventional RF ablation (RF: radiofrequency), in which the tissue temperature increases by 20° C. to 70° C. and cells are destroyed by heat. In IRE, bipolar pulses are generally used, that is to say a combination of positive and negative pulses, in order to avoid as far as possible muscle contractions, which usually occur in the case of the application of DC voltage. These pulses can be applied between two bipolar electrodes of a catheter or between a catheter electrode and a body surface electrode, which is usually applied to the patient's back.

In order that the IRE pulses generate the desired pores in the tissue, the electrical field strength E, defined by the pulses, at the tissue between a pair of at least two electrodes must exceed a tissue-dependent threshold value Eth. For example, the threshold value for cardiac cells is approximately 500 V/cm, while for bones it is 3000 V/cm. These differences in the threshold field strengths allow the selective application of IRE in different tissue. In order to achieve the required field strength, the voltage to be applied to an electrode pair depends both on the target tissue and also on the distance between the electrodes and on the size of the electrodes themselves. These parameters likewise also influence the thermal energy input during the ablation and thus the temperature peaks which can occur at the tissue to be treated. The applied voltages can reach up to 2000 V, which is substantially higher than the voltages of 10-200 V that are typical in the case of thermal RF ablation.

The bipolar pulsed field ablation pulse (bipolar PFA pulse) for IRE comprises a positive and a negative pulse, which are applied between two electrodes with a pulse width of from 1 to 5 μs and an interval between the positive and negative pulses of from 1 to 5 μs. The bipolar pulses are combined to form pulse sequences, wherein each sequence can comprise over one hundred bipolar pulses with a pulse-to-pulse interval of from 1 to 10 ms. The pulse sequences in each case form a burst, wherein the entire pulse packet of the IRE ablation is composed of from 1 to 20 bursts/burst units, each of which has a burst-to-burst interval of from 1 to 1000 ms. The total duration of an ablation can be up to 10 s.

Document US 2022/019274 A1 discloses a spherical structure having a plurality of elongate elements, on each of which a plurality of converters is arranged.

Document US 2021/0169567 A1 discloses a shaft of a catheter which is adapted to be inserted into an organ of a patient. An expandable balloon is connected to the distal end of the catheter. A plurality of electrodes is arranged on an outer side of a membrane of the balloon.

Document WO 2022/256218 A1 discloses a catheter having a catheter shaft and a balloon. The balloon is arranged at a distal end region of the catheter shaft. One or more electrodes are arranged on an outer surface or an inner surface of the balloon.

Document CN 216495608 U discloses a balloon-shaped catheter on the outer sides of which a plurality of electrodes is arranged.

Document US 2022/0222954 A1 discloses a balloon catheter having a plurality of electrodes arranged thereon.

Document WO 2019/181634 A1 discloses a balloon catheter having a plurality of electrodes, which are arranged around the balloon.

Document WO 2021/116774 A1 discloses a balloon catheter having a plurality of electrodes arranged at a distal end of the balloon catheter.

Document US 2021/0153935 A1 discloses a balloon catheter around the outer shell of which an electrode is wrapped. A loop catheter protrudes helically from the distal endpiece of the balloon catheter, on which loop catheter electrodes are arranged.

There are further known documents US 2022/0233236 A1, EP 3 456 278 A2 and US 2022/0241008 A1.

### SUMMARY

The present invention addresses the problem of time-reduced electroporation.

To this end, a device according to claim 1 and a method according to claim 13 are proposed.

According to a first aspect, a device for the irreversible electroporation of a tissue of a patient is proposed. The device has a catheter. The catheter has a proximal end, and a (e.g. single), in particular separate/single, distal electrode arranged at a distal, in particular outermost, end of the catheter (tip electrode). The catheter has a membrane arranged between the distal end and the proximal end. The membrane is adapted to assume a first, in particular tubular or collapsed, form state and a second, in particular balloon-shaped or expanded, form state. The catheter has a plurality of electrodes, in particular (spatially) separate from the distal electrode. The plurality of electrodes is arranged on the membrane. The device has a signal generator-evaluation unit connected to the proximal end of the catheter and adapted to carry out a tissue impedance determination, in particular a local tissue impedance determination, and/or an ablation.

The signal generator-evaluation unit can have a signal generator unit and/or an evaluation unit and/or a control unit. The signal generator-evaluation unit can be adapted to carry out a tissue impedance determination or an ablation, or a tissue impedance determination and, for example subsequently, an ablation.

The device can further have a proximal electrode arranged at the proximal end of the catheter. In addition or alternatively, the device can have at least one additional electrode, which is arranged between the membrane and the distal electrode.

The signal generator-evaluation unit can be adapted to deliver a first electrical signal into the tissue via an electrode constellation. The signal generator-evaluation unit can be adapted to deliver a first electrical signal into the tissue and to receive a second electrical signal from the tissue via the electrode constellation. The electrode constellation can be understood as being a pairwise association of electrodes. The signal generator-evaluation unit can be adapted to actuate electrode pairs and/or electrode constellations and/or to switch between electrodes (electrode pairs) and/or electrode constellations.

An electrode constellation can be defined by/formed by/composed of the proximal and distal electrodes as a first electrode pair and the additional electrode and the distal electrode as a second electrode pair, in particular in the first form state.

An electrode constellation can be defined by/formed by/composed of the proximal and distal electrodes as a first electrode pair and one of the plurality of electrodes and the additional electrode, in particular in the second form state, as a second electrode pair.

An electrode constellation can be defined by/formed by/composed of two electrodes, arranged next but one to one another, of the plurality of electrodes as a first electrode pair and the distal electrode and a further electrode, in particular situated between the two electrodes arranged next but one to one another, of the plurality of electrodes, in particular in the second form state, as a second electrode pair

Electrodes arranged next but one to one another can be understood as being electrodes or an electrode pair between which there is a common, directly (immediately) adjacent electrode.

In a first operating phase, the first electrical signal can be configured as an electrical current signal and the second electrical signal can be configured as an electrical voltage signal. In the first operating phase, the signal generator-evaluation unit can be adapted to determine at least one, in particular local, tissue impedance from the electrical current signal transmitted into the tissue (=the first electrical signal) and the electrical voltage signal received from the tissue (=second electrical signal).

The signal generator-evaluation unit can be adapted to determine at least two, in particular local, tissue impedances by means of at least two electrode constellations/electrode pairs/electrodes (to be actuated), in particular chosen/switched in (temporal) succession.

The signal generator-evaluation unit can be adapted to actuate, within an electrode constellation, at least one other first and/or second electrode pair, in particular in temporal succession, and to determine at least two, in particular local, tissue impedances for the electrode constellation. In other words, the signal generator-evaluation unit can be adapted to actuate, within a chosen electrode constellation, other electrodes within the first and/or second electrode pair, whereby a new first and/or new second electrode pair is formed, with which, in particular owing to a different signal path through the tissue, a tissue impedance, in particular a local tissue impedance, can be determined.

The signal generator-evaluation unit can be adapted, for (an electrode constellation) the proximal and distal electrodes as the first electrode pair and one of the plurality of electrodes and the additional electrode, in particular in the second form state, as the second electrode pair, to actuate the second electrode pair at least a single time from at least one further electrode of the plurality of electrodes and the additional electrode, in particular in the second form state, and to determine at least one further, in particular local, tissue impedance.

In other words, for the one electrode constellation, the first electrode pair can be formed by the proximal and distal electrodes and the second electrode pair can be formed by one of the plurality of electrodes and the additional electrode. The signal generator-evaluation unit can be adapted, after each measurement of the second electrical signal/electrical voltage signal, to actuate a further electrode of the plurality of electrodes and the additional electrode until a second electrical signal/electrical voltage signal has been measured across all possible electrode combinations of the plurality of electrodes and the additional electrode.

The signal generator-evaluation unit can be adapted to form for (an electrode constellation) two electrodes, arranged next but one to one another, of the plurality of electrodes as the first electrode pair and the distal electrode and a further electrode, in particular situated between the two electrodes arranged next but one to one another, of the plurality of electrodes, in particular in the second form state, as the second electrode pair. The signal generator-evaluation unit can be adapted to actuate/to form the first electrode pair at least a single time from two further electrodes, arranged next but one to one another, of the plurality of electrodes, in particular in the second form state, and the second electrode pair from the distal electrode and a further electrode, in particular situated between the two further electrodes arranged next but one to one another, of the plurality of electrodes, in particular in the second form state, and to determine at least one further, in particular local, tissue impedance.

In a second operating phase, the electrical signal can be configured as an electrical voltage signal. The signal generator-evaluation unit can be adapted to generate the electrical voltage signal in accordance with a burst-signal protocol to be chosen and to transmit said signal into the tissue via a first electrode pair. The signal generator-evaluation unit can, in particular in the second operating phase, actuate one electrode pair.

The first electrode pair can, in particular in a second form state, be formed by the distal electrode and one of the plurality of electrodes. The first electrode pair can, in particular in a second form state, be formed by two, in particular (immediately) adjacent, electrodes of the plurality of electrodes.

The device can have a counter electrode, in particular a body surface counter electrode, connected to the signal generator-evaluation unit. The first electrode pair can be formed by one of the plurality of electrodes and the counter electrode, in particular in the second form state. The first electrode pair can, in particular in the first form state, be formed by the distal electrode and the counter electrode.

In a first operating phase, the first electrical signal can be configured as an electrical current signal and the second electrical signal can be configured as an electrical voltage signal. In a first operating phase, the signal generator-evaluation unit can be adapted to determine at least one, in particular global and/or local, tissue impedance from the electrical current signal transmitted into the tissue and the electrical voltage signal received from the tissue. The first electrical signal can be transmitted into the tissue via a first electrode pair. The second electrical signal can be received via the first electrode pair. In other words, an electrical current can be transmitted into the tissue and an electrical voltage can be received via the same electrode pair.

The signal generator-evaluation unit can be adapted, within an electrode pair or starting from an electrode pair, to switch, in particular in temporal succession, to at least one further electrode of the plurality of electrodes. In other words, one electrode of the electrode pair can be replaced by a further of the plurality of electrodes. In this way, a new electrode pair can be formed. This can be carried out repeatedly, in particular in temporal succession.

The electrode(s) (in particular the plurality of electrodes, the proximal, the distal and/or the additional electrode) can be connected to the signal generator-evaluation unit via the proximal end of the catheter by means of electrical line(s) which are insulated from one another and with respect to the immediate surroundings and which in particular are arranged externally or internally on/in the catheter.

The signal generator-evaluation unit can be adapted, in particular in a first and/or second operating phase, to generate a first electrical signal on the basis of a burst-signal protocol and to transmit/send said signal to a first electrode pair.

Formats of the burst-signal sequence protocol can specify properties and the amount of energy per burst. The format(s) can have: a first number of bursts within the burst-signal sequence, at least one first time interval between at least two successive bursts of the burst-signal sequence, a second number of bipolar pulses within a burst, at least one second time interval between at least two successive bipolar pulses within a burst, a third time interval between a positive and a negative pulse of at least one bipolar pulse, a pulse width of a positive and/or negative pulse of at least one bipolar pulse, and/or a value of a pulse deflection of a positive and/or negative pulse of at least one bipolar pulse.

A first number of bursts within the burst-signal sequence can lie in a value range of from 1 to 100 burst units.

At least one first time interval between two successive bursts of the burst-signal sequence can lie in a value range of from 1 ms to 1000 ms. A second number of bipolar pulses within a burst can lie in a value range of from 1 to 300 bipolar pulse units.

At least one second time interval between at least two successive bipolar pulses within a burst can lie in a value range of from 1 to 10 ms. A third time interval between a positive and a negative pulse can lie in a value range between 1 and 5 μs.

A pulse width of a positive and/or negative pulse can lie in a value range between 1 and 10 μs. A pulse width of a positive pulse can be different from a pulse width of a negative pulse.

A value of a pulse deflection of a positive pulse can lie in a value range of from 200 to 2000 V. A value of a pulse deflection of a negative pulse can lie in a value range of from −200 to −2000 V.

According to a second aspect, a method for the irreversible electroporation of a tissue of a patient is proposed. The method comprises providing a catheter. The catheter has a proximal end and a distal electrode arranged at a distal end of the catheter. The catheter has a membrane arranged between the distal end and the proximal end. The membrane is adapted to assume a first or second form state. The catheter has a plurality of electrodes, which are arranged on the membrane. The method comprises providing a signal generator-evaluation unit connected to the proximal end of the catheter. The method comprises carrying out a tissue impedance determination and/or carrying out an ablation. In other words, the method comprises (i) carrying out a tissue impedance determination or (ii) carrying out an ablation or (iii) carrying out a tissue impedance determination and carrying out an ablation, for example carrying out an ablation subsequent to or following the tissue impedance determination.

Further features, properties, advantages and possible modifications will become clear to a person skilled in the art from the following descriptions, in which reference is made to the accompanying drawings.

### BRIEF DESCRIPTION OF THE DRAWINGS

FIG. 1 is a schematic representation of a bipolar IRE pulse according to an exemplary embodiment of the invention.

FIG. 2 is a schematic representation of a pulse protocol having multiple series or bursts of bipolar pulses according to an exemplary embodiment of the invention.

FIG. 3 shows schematic representations of a device with an expanded balloon membrane.

FIG. 4 shows schematic representations of the device from FIG. 3 with the membrane folded in in the focal configuration.

FIG. 5 shows schematic connections of the electrodes for local impedance measurement for the focal catheter configuration.

FIG. 6 shows schematic connections of the electrodes for local impedance measurement for the “one-shot” catheter configuration.

FIG. 7 shows schematic further connections of the electrodes for local impedance measurement for the “one-shot” catheter configuration.

FIG. 8 shows, schematically, the possible ablation modes of the device in the expanded form state of the membrane.

FIG. 9 shows, schematically, the possible ablation modes of the device for the collapsed form state of the membrane.

FIG. 10 shows, schematically, the measurement of a global tissue impedance of the device in an expanded state of the membrane.

FIG. 11 shows, schematically, the measurement of a global tissue impedance of the device in a collapsed state of the membrane.

### DETAILED DESCRIPTION OF THE EMBODIMENTS

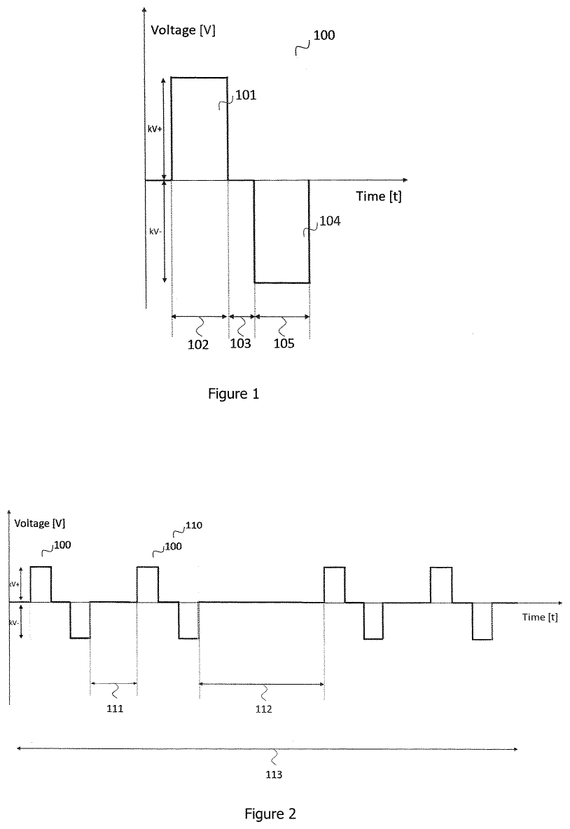

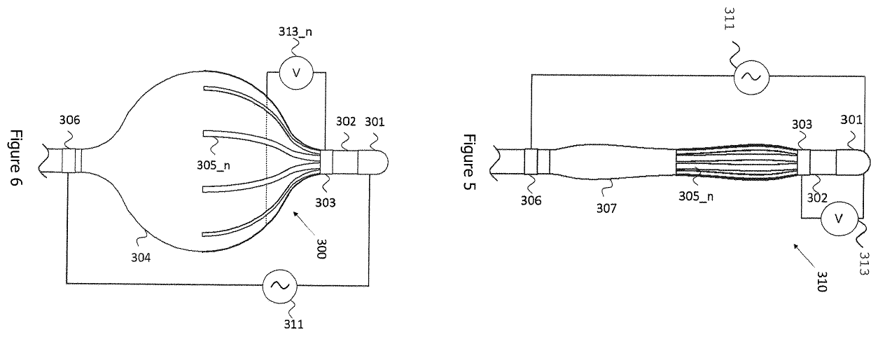

FIG. 1 shows a schematic representation of a bipolar pulse 100 which is generated by the signal generator-evaluation unit when the signal generator-evaluation unit generates a first electrical signal in accordance with a burst-signal sequence protocol in the first operating phase of a device. In the present example, the formats, which determine the properties of the bipolar pulse 100, have been predefined by a user. The values of the pulse deflection kV+, kV− of the positive pulse 101 and of the negative pulse 104 are ±500 kV in the example shown. The third time interval 103 between the positive pulse 101 and the negative pulse 104 is 2.5 μs. The pulse width 102 of the positive pulse 101 differs from the pulse width 105 of the negative pulse 104. The difference in the pulse widths is not shown in FIG. 1. For an explanation of the tissue impedance measurement with the electrode constellation(s) actuated by the signal generator-evaluation unit, reference is made to FIGS. 4 to 9.

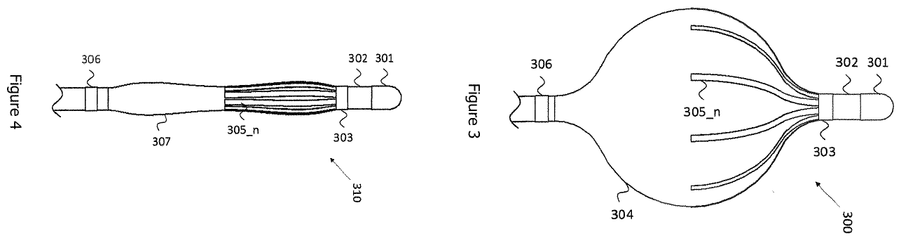

FIG. 2 shows, schematically, a first electrical signal in a second operating phase of the device. The first electrical signal is configured as a burst-signal sequence. Two bursts can be seen, one of which is provided with the reference numeral 110. Each burst has two bipolar pulses 100. Each bipolar pulse 100 that occurs in the burst-signal sequence has the properties of the format from the preceding figure description. The first number of bursts, here by way of example two bursts, the second time interval 111, and the first time interval 112 between two successive bursts 110 have been defined by a user before a first operating phase. The burst-signal sequence that can be seen extends over a duration 113, which corresponds to the duration of the irreversible electroporation.

FIG. 3 shows schematic representations of the device, in which the membrane 304 is expanded. The device is in a “one-shot” configuration 300. The device has a shaft 302, which can be in a controllable or non-controllable configuration, and a filled membrane 304 permanently fastened thereto. Controllable refers, for example, to the possibility of imparting a bend to the device by means of a handle arranged on the shaft by a rotational movement of the handle and steering the device in the organ of a patient. A distal electrode 301 and an additional electrode 303 are arranged on the shaft 302. The additional electrode 303 is arranged distally to the membrane 304. A proximal electrode 306 is arranged proximally from the membrane 304. The electrodes 301, 303 and 306 are electrically contacted via one or more lines, which extend from the proximal end over the shaft to the electrodes and are electrically insulated with respect to one another. A plurality of electrodes 305_n is arranged on an outer surface of the membrane 304, where n here corresponds to the number of electrodes and there can be, by way of example and without being limited thereto, up to 15 electrodes. These are electrically connected via one or more strip conductors, which extend from the proximal end of the device over the shaft to the electrodes 305_n. The one or more strip conductors are covered such that they are electrically insulated both from one another and with respect to the external environment.

FIG. 4 shows schematic representations of the device, in which the membrane 304 is collapsed/folded. The device is in a focal configuration 310. The device has a shaft 302, which can be in a controllable or non-controllable configuration, and a membrane 307 in collapsed form permanently fastened thereto. A distal electrode 301 and an additional electrode 303 are arranged on the shaft 302. The additional electrode 303 is arranged distally to the membrane 304. A proximal electrode 306 is arranged proximally from the membrane 304. The electrodes 301, 303 and 306 are electrically contacted, wherein these wires are guided via electrical lines to the proximal end of the device and are electrically insulated from one another. A plurality of electrodes 305_n is arranged on an outer surface of the membrane 307, where n here corresponds to the number of electrodes and there can be, by way of example and without being limited thereto, up to 15 electrodes. These are electrically connected via one or more strip conductors, which extend from the proximal end of the device over the shaft to the electrodes. The one or more strip conductors are covered such that they are electrically insulated both from one another and with respect to the external environment.

FIG. 5 shows the device in the focal configuration 310 for actuating the electrodes and for determining the local impedance of the tissue. The device has a distal electrode 301, an additional electrode 303 and a proximal electrode 306. The membrane 307 arranged between the additional electrode 303 and the proximal electrode 306 is in a first form state; the membrane is collapsed. A plurality of electrodes 305_n is arranged on the membrane 307. The first electrical signal is here configured as an electrical current signal 313. The second electrical signal is configured as an electrical voltage signal. The signal generator-evaluation unit (not shown here) actuates the corresponding electrode constellation with the corresponding electrode pairs.

The electrical signal 311 is applied between the distal electrode 301, also called the tip electrode, and the proximal electrode 306 and forms the first electrode pair, via which the electrical current is transmitted into the tissue, which is in contact with the electrode pair. An electrical voltage signal 315 is measured via a second electrode pair, here the distal electrode 301 and the additional electrode 303. The first electrode pair formed by the electrodes 301, 306 and the second electrode pair formed by the electrodes 301, 303 together form an electrode constellation. The local tissue impedance can be determined by the relationship of Ohm's law on the basis of the electrical current transmitted via the first electrode pair and the electrical voltage received via the second electrode pair.

FIG. 6 shows the device in the “one-shot” configuration 300 for actuating the electrodes and for determining the local tissue impedance of the tissue. The device has a distal electrode 301, an additional electrode 303 and a proximal electrode 306. The membrane 304 arranged between the additional electrode 303 and the proximal electrode 306 is in a second form state; the membrane 304 is expanded. A plurality of electrodes 305_n is arranged on the membrane 304. The first electrical signal is here configured as an electrical current signal 311. The second electrical signal is configured as an electrical voltage signal 311. The signal generator-evaluation unit (not shown here) actuates the corresponding electrode constellation with the corresponding electrode pairs in order to transmit current into the tissue and to measure voltage at the tissue via the electrode constellation.

The electrical current 311 is applied between the distal electrode 301 and the proximal electrode 306 (first electrode pair). In the present case, the proximal electrode 306 is configured as a ring electrode. For tissue impedance determination by means of Ohm's law, voltages 313_n are measured from each of the plurality of electrodes 305_n to the additional electrode 303 (second electrode pair). To this end, once a measurement has been made between one of the plurality of electrodes 305_n and the additional electrode 303, the signal generator-evaluation unit controls/switches to a further electrode of the plurality of electrodes 305_n until all of the plurality of electrodes 305_n have been actuated for a measurement of the electrical voltage 313_n between the plurality of electrodes 305_n and the additional electrode 303. Here, n again corresponds to the number of electrodes on the membrane 304, so that the same number of determined local impedances is accordingly also obtained and the properties of the target tissue can be determined very selectively.

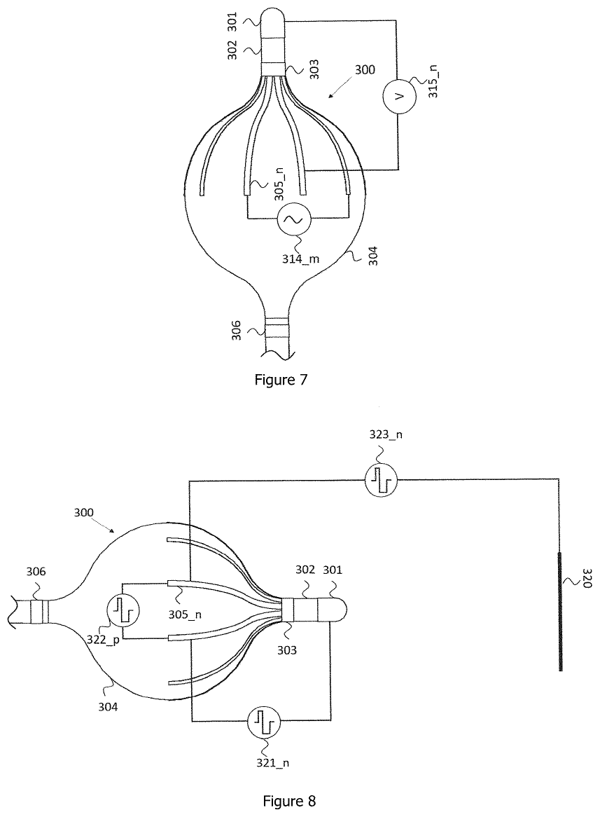

FIG. 7 shows, schematically, the device in the “one-shot” configuration 300 for actuation of the electrodes and for determination of the local tissue impedance of the tissue. The device has a distal electrode 301, an additional electrode 303 and a proximal electrode 306. The membrane 304 arranged between the additional electrode 303 and the proximal electrode 306 is in a second form state; the membrane 304 is expanded. A plurality of electrodes 305_n is arranged on the membrane 304. The first electrical signal is here configured as an electrical current signal 314_m. The second electrical signal is configured as an electrical voltage signal 315_n. The signal generator-evaluation unit (not shown here) actuates the corresponding electrode constellation with the corresponding electrode pairs.

The electrical current 314_m is applied between two electrodes, arranged next but one to one another, of the plurality of electrodes 305_n (first electrode pair). For the tissue impedance determination by means of Ohm's law, the voltage 315_n is measured between one electrode of the plurality of electrodes and the distal electrode 301 (second electrode pair). The one electrode of the plurality of electrodes of the second electrode pair is the electrode that is shared as the immediately adjacent electrode by those electrodes of the plurality of electrodes 305_n of the first electrode pair that are arranged next but one to one another. The voltage is then measured between the one electrode of the plurality of electrodes 305_n of the second electrode pair and the distal electrode 301 and the tissue impedance is determined by Ohm's law.

The signal generator-evaluation unit is adapted, for example by means of a multiplexer circuit, to apply the electrical current 314_m in succession between all the possible electrodes arranged next but one to one another, so that one electrode of the plurality of electrodes is always omitted, to which the voltage is measured.

In the present case, the plurality of electrodes 305_n is configured by way of example as 15 electrodes. By means of the actuation described above, when the electrical current has been applied in succession to all the possible electrodes arranged next but one to one another and the electrical current has been measured across the omitted electrode, 15 local tissue impedances can be determined from the measured values.

FIG. 8 shows the device for the “one-shot” configuration 300 for actuation of the electrodes and for delivery of IRE pulses. The device has a distal electrode 301, an additional electrode 303, a proximal electrode 306 and a counter electrode 320. The membrane 304 arranged between the additional electrode 303 and the proximal electrode 306 is in a second form state; the membrane is expanded. A plurality of electrodes 305_n is arranged on the membrane 304. The first electrical signal is here configured as an electrical voltage signal (321_n, 322_p, 323_n).

The signal generator-evaluation unit (not shown here) actuates a first electrode pair, via which the ablation is carried out. In the schematic representation, three (1)(2)(3) first electrode pairs are shown, via which the ablation is carried out: (1) two electrodes of the plurality of electrodes 305_n, (2) one of the plurality of electrodes and the distal electrode 301, and (3) one of the plurality of electrodes and the counter electrode 320.

If the signal generator-evaluation unit actuates the third (3) first electrode pair, delivery of the IRE pulses 323_n takes place via that electrode pair, and the resulting electrical field forms between in each case one of the plurality of electrodes 305_n and the counter electrode 320. By means of the signal generator-evaluation unit, which to this end has, for example, a multiplexer circuit, all the further electrodes of the plurality of electrodes 305_n are successively actuated, so that each of the plurality of electrodes 305_n, after the ablation procedure has been carried out, was active at least once and the IRE pulses 323_n could in each case be delivered to the counter electrode.

If the signal generator-evaluation unit actuates the second (2) first electrode pair, the ablation procedure proceeds as in the preceding paragraph, but the IRE pulses 321_n are in each case delivered to the distal electrode 301.

If the signal generator-evaluation unit actuates the first (1) first electrode pair, the IRE pulses 322_p are delivered via that electrode pair, and the resulting electrical field forms between two immediately adjacent electrodes of the plurality of electrodes 305_n.

By means of the signal generator-evaluation unit, which to this end has, for example, a multiplexer circuit, all the electrodes are successively connected through in pairs until all the electrodes were active at least once.

For an iterative through-connection of the electrodes of the third first electrode pair, it is the case that a first electrode pair from a current iteration of the actuation/switching process has an electrode of the plurality of electrodes in common with a first electrode pair of the preceding iteration. Owing to the multiplexing, all three electrode pairs allow a circular lesion pattern encircling the pulmonary vein to be produced. Actuation of the electrode pairs (1) and (2) represents in each case a monopolar ablation configuration. Ablation via the third (3) first electrode pair represents a bipolar ablation configuration.

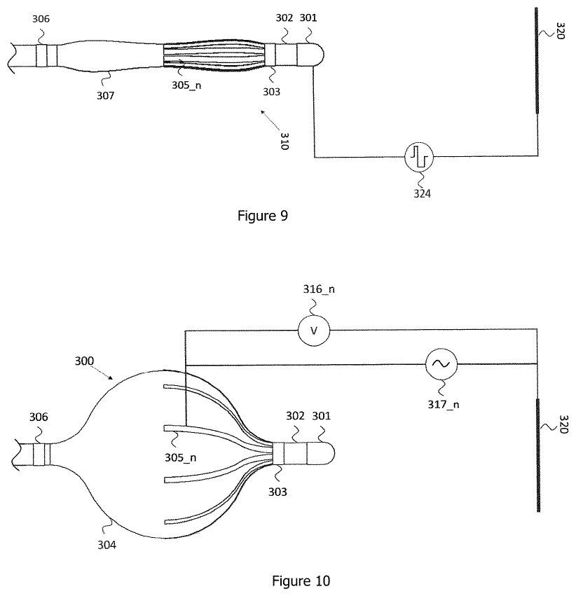

FIG. 9 shows the device in the focal configuration 310 for actuation of an electrode pair for IRE pulse delivery. Here too, a body surface electrode 320 is used, said electrode serving as the counter-pole to the distal electrode 301. The IRE pulses 324 can thus be delivered. This ablation represents a monopolar ablation configuration.

FIG. 10 shows, schematically, the measurement of a global impedance of the device in an expanded state of the membrane for determination of the global tissue impedance of the tissue. The device has a distal electrode 301, an additional electrode 303, a proximal electrode 306 and a counter electrode 320. The membrane 304 arranged between the additional electrode 303 and the proximal electrode 306 is in a second form state; the membrane 304 is expanded. A plurality of electrodes 305_n is arranged on the membrane 304. The first electrical signal is here configured as an electrical current signal 317_n. The second electrical signal is configured as an electrical voltage signal 316_n. The signal generator-evaluation unit (not shown here) actuates a first electrode pair. The first electrode pair is formed by an electrode of the plurality of electrodes 305_n and the counter electrode 320. The electrical current is sent into the tissue and the electrical voltage is measured via the first electrode pair. The signal generator-evaluation unit (not shown) determines a global tissue impedance from the electrical current and the electrical voltage by Ohm's law.

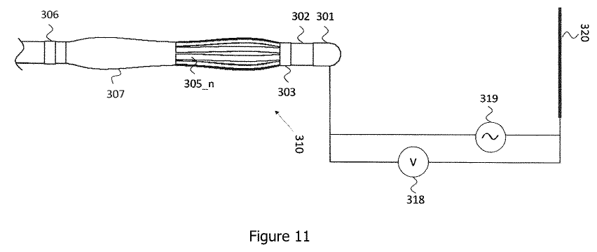

FIG. 11 shows, schematically, the measurement of a global impedance of the device in a collapsed state of the membrane. The device has a distal electrode 301, an additional electrode 303, a proximal electrode 306 and a counter electrode 320. The membrane 304 arranged between the additional electrode 303 and the proximal electrode 306 is in a first form state; the membrane 304 is collapsed. A plurality of electrodes 305_n is arranged on the membrane 304. The first electrical signal is here configured as an electrical current signal 319. The second electrical signal is configured as an electrical voltage signal 318. The signal generator-evaluation unit (not shown here) actuates a first electrode pair. The first electrode pair is formed by the distal electrode 301 and the counter electrode 320. The electrical current is sent into the tissue and the electrical voltage is measured via the first electrode pair. The signal generator-evaluation unit (not shown) determines a global tissue impedance from the electrical current and the electrical voltage by Ohm's law.

What is claimed is:

1. A device for the irreversible electroporation of a tissue of a patient, said device comprising:
a catheter including:
a proximal end, and a distal electrode arranged at a distal end of the catheter;
a membrane arranged between the distal end and the proximal end and adapted to assume a first and a second form state;
a plurality of electrodes arranged on the membrane; and
a signal generator-evaluation unit coupled to the proximal end of the catheter and adapted to carry out a tissue impedance determination and/or an ablation.

2. The device according to claim 1, further comprising:
a proximal electrode arranged at the proximal end of the catheter; and/or
at least one additional electrode arranged between the membrane and the distal electrode.

3. The device according to claim 1, wherein the signal generator-evaluation unit is adapted to deliver a first electrical signal into the tissue via an electrode constellation, or to deliver a first electrical signal into the tissue and to receive a second electrical signal from the tissue via the electrode constellation.

4. The device according to claim 3, wherein the electrode constellation includes:
proximal and distal electrodes as a first electrode pair and an additional electrode and the distal electrode as a second electrode pair in the first form state; or
the proximal and distal electrodes as a first electrode pair and one of the plurality of electrodes and the additional electrode as a second electrode pair in the second form state; or
two electrodes of the plurality of electrodes arranged next to each other as a first electrode pair and the distal electrode and a further electrode situated between the two electrodes arranged next to each other as a second electrode pair in the second form state.

5. The device according to claim 3, wherein in a first operating phase:
the first electrical signal is an electrical current signal and the second electrical signal is an electrical voltage signal; and
the signal generator-evaluation unit is adapted to determine at least one local tissue impedance from the electrical current signal transmitted into the tissue and the electrical voltage signal received from the tissue.

6. The device according to claim 5, wherein the signal generator-evaluation unit is adapted to determine at least two local tissue impedances by at least two electrode constellations that are chosen/switched in temporal succession.

7. The device according to claim 2, wherein the signal generator-evaluation unit is adapted to actuate, within an electrode constellation, in temporal succession, at least one other first and/or second electrode pair and to determine for the electrode constellation at least two local tissue impedances.

8. The device according to claim 4, wherein the signal generator-evaluation unit is adapted for the proximal and distal electrodes as the first electrode pair and one of the plurality of electrodes and the additional electrode, in the second form state, as the second electrode pair, to actuate the second electrode pair at least a single time from at least one further electrode of the plurality of electrodes and the additional electrode in the second form state, and to determine at least one further local tissue impedance, and/or
the signal generator-evaluation unit is adapted for two electrodes, arranged next to each other, as the first electrode pair and the distal electrode and a further electrode situated between the two electrodes arranged next to each other in the second form state, as the second electrode pair, to actuate the first electrode pair at least a single time from two further electrodes, arranged next to each other, in the second form state, and the distal electrode and a further electrode, situated between the two further electrodes arranged next to each other in the second form state, as the second electrode pair, and to determine at least one further local tissue impedance.

9. The device according to claim 5, wherein in a second operating phase:
the electrical signal is configured as an electrical voltage signal; and
the signal generator-evaluation unit is adapted to generate the electrical voltage signal in accordance with a burst-signal protocol to be chosen and to transmit said signal into the tissue via a first electrode pair; wherein
the distal electrode and one of the plurality of electrodes, in the second form state, is configured as the first electrode pair, or
two adjacent electrodes of the plurality of electrodes, in the second form state, are configured as the first electrode pair.

10. The device according to claim 1, further comprising a body surface counter electrode connected to the signal generator-evaluation unit, wherein:
one of the plurality of electrodes and the counter electrode, in the second form state, are configured as a first electrode pair; and/or
the distal electrode and the counter electrode in the first form state is configured as the first electrode pair; and/or
in a first operating phase:
the first electrical signal is configured as an electrical current signal and the second electrical signal is configured as an electrical voltage signal; and
the signal generator-evaluation unit is adapted to determine at least one global tissue impedance from the electrical current signal transmitted into the tissue, via the first electrode pair, and the electrical voltage signal received from the tissue, via the first electrode pair.

11. The device according to claim 9, wherein the signal generator-evaluation unit is adapted, within an electrode pair, to switch, in temporal succession, to at least one further electrode of the plurality of electrodes.

12. The device according to claim 2, wherein the proximal, the distal and the additional electrodes are connected to the signal generator-evaluation unit via the proximal end of the catheter by electrical lines that are insulated from one another and with respect to the immediate surroundings and that are arranged externally on the catheter.

13. A method for the irreversible electroporation of a tissue of a patient, comprising the steps:
providing a catheter including:
a proximal end, and a distal electrode arranged at a distal end of the catheter;
a membrane arranged between the distal end and the proximal end and adapted to assume a first and a second form state;
a plurality of electrodes arranged on the membrane;
providing a signal generator-evaluation unit connected to the proximal end of the catheter; and
carrying out a tissue impedance determination or carrying out a tissue impedance determination and subsequently carrying out an ablation.
